(19) United States
(12) Patent Application Publication
(10) Pub. No.: US 2026/0256901 A1
(43) Pub. Date: Sep. 3, 2026

(54) ADMINISTRATION OF BACTERIAL COMPOSITION FOR USE IN TREATMENT OF LUNG METASTASES

(71) Applicant: Immuvera Therapeutics AS, Oslo (NO)

(72) Inventors: Francesca Gatti, Oslo (NO); Mikael Mathias Kolmannskog Kerboeuf, Oslo (NO)

(21) Appl. No.: 19/489,481

(22) PCT Filed: Jun. 7, 2024
(86) PCT No.: PCT/EP2024/065753

§ 371 Date: Dec. 2, 2025

(30) Foreign Application Priority Data
Jun. 7, 2023 (GB) 2308459.3

(51) Int. Cl. A61K 39/04; A61D 7/04; A61K 39/00; A61P 35/04

(52) U.S. Cl. CPC A61K 39/04; A61D 7/04; A61P 35/04; A61K 2039/552; A61K 2039/585; A61K 2039/86

(57) ABSTRACT

The present invention relates to an administration procedure to effectively treat a companion animal suffering from lung metastases. In particular, a nebulized BCG composition is delivered directly to the lungs to increase uptake therein and avoid loss of the active ingredient e.g. in the mucosa of the upper airways.

### TECHNICAL FIELD OF THE INVENTION

The present invention relates to an administration procedure to effectively treat a companion animal suffering from lung metastases. In particular, a nebulized BCG composition is delivered directly to the lungs to increase uptake therein and avoid loss of the active ingredient e.g. in the mucosa of the upper airways.

### BACKGROUND OF THE INVENTION

The Bacillus Calmette-Guérin (BCG) vaccine comprises viable bacteria of attenuated Mycobacterium bovis. This vaccine has been used for a long time as prophylaxes against tuberculosis (Mycobacterium tuberculosis) in humans. More recently, BCG vaccine has also shown to be an effective treatment for some types of tumours in humans.

The mechanism of action of BCG in the treatment of cancer is broad and not fully elucidated. Components from the viable bacteria are well known agonists/activators of pattern recognition receptors (PRR) expressed by antigen presenting cells (dendritic cells, macrophages and monocytes). When these receptors (toll-like receptors and NOD-receptors) are activated, this induces a downstream production of pro-inflammatory cytokines, such as TNF-α, IL-1b, IL-2, IL-6, and IL-12. These cytokines can then stimulate a tumour-targeted cytotoxic T-cell and NK cell responses, which results in the elimination of the cancer cells. In the tumour tissue, the cancer cells create an immunological barrier which establishes an immunosuppressive and tumour permissive environment. BCG can on the other hand break this barrier, by being taken up by macrophages, in which the tumour tissue is rich (tumour associated macrophages) and skew the immunosuppressive tumour microenvironment towards a pro-inflammatory state.

As for humans, BCG has been shown to induce a positive clinical response in dogs affected by certain types of cancer. Encouragingly, several routes of administration, including intradermal, subcutaneous, intramuscular, intratumoural and intravenous, have been demonstrated to be safe with low occurrence of serious side effects. However, lung metastases remain elusive and difficult to treat. One challenge with lung metastases is to deliver any active ingredient effectively to the site of disease in the lower airways. Consequently, the prognosis for companion animals, such as dogs, which develops lung metastases from a primary solid tumour is very poor.

As an example, osteosarcoma (OS) is a highly malignant bone tumour in dogs. Despite radical surgical treatment, over 90% of dogs develop pulmonary metastases. Most dogs with OS die as result of these metastases. As of today, there are no effective treatment options for such metastases, once they have developed.

Accordingly, there is a great need to provide an effective treatment option for lung metastases in companion animals, such as dogs.

In particular, it would be advantageous to provide a treatment which effectively delivers BCG to the site of the lung metastases of the patient, with minimal systemic exposure and adverse effects.

### SUMMARY OF THE INVENTION

The present invention relates to an improved treatment of lung metastases in companion animals. The treatment includes administration of BCG to the lungs of the patient, such as by intubation, and further be adjusted by conditions of positive pressure ventilation. The treatment has surprisingly been shown to be efficient in a clinical setting and may offer a solution to patients for which there are currently no viable treatments.

Thus, an object of the present invention relates to a treatment of lung metastases in a companion animal.

In particular, it is an object of the present invention to provide a more efficient method for administration of a BCG composition to a companion animal suffering from lung metastases.

Thus, an aspect of the present invention relates to a Bacillus Calmette-Guérin (BCG) composition for use in the treatment, inhibition or amelioration of lung metastases in a companion animal, wherein the BCG composition is administered to the lungs of a companion animal.

An embodiment of the present invention relates to the BCG composition for use as described herein, wherein the BCG composition is administered to the lungs via a tracheal tube.

Another embodiment of the present invention relates to the BCG composition for use as described herein, wherein the BCG composition is administered under conditions of positive pressure ventilation of the companion animal.

### BRIEF DESCRIPTION OF THE FIGURES

FIG. 1 shows X-ray images of the lungs of canine patient X. Comparison of a lung metastasis (white arrow) derived from a melanoma tumour before the first administration of BCG on day 0 (A) and before the fourth BCG administration on day 33 (B) shows a visual reduction of the diameter of the metastatic tissue.

FIG. 2 shows CT scans of the lungs of canine patient X. Comparison of a lung metastasis (white arrow) derived from a melanoma tumour before the first administration of BCG on day 0 (A) and before the fourth BCG administration on day 33 (B) shows a visual reduction of the diameter of the metastatic tissue.

FIG. 3 shows a zoom of the CT scans of FIG. 2. Software is utilised to measure the size of the lung metastasis (A) before the first administration of BCG on day 0 and (B) before the fourth BCG administration on day 33.

FIG. 4 shows plasma secretion of (A) interleukin 8 (IL-8) and (B) interleukin 10 (IL-10) in the canine patient X before and following treatment with BCG. IL-8 is a marker of disease burden in melanoma and correlates with prognoses, where lower amounts of plasma IL-8 correlates with better prognoses. IL-10 is an anti-inflammatory cytokine, with decreasing levels indicating reduced immunosuppression.

The present invention will in the following be described in more detail.

### DETAILED DESCRIPTION OF THE INVENTION

### Definitions

Prior to outlining the present invention in more details, a set of terms and conventions is first defined:

### Primary Solid Tumour

In the present context, the term “primary solid tumour” refers to an abnormal mass of tissue that grows uncontrollably. The primary solid tumour is the original, or first, in the body. The primary solid tumour may spread to other parts of the body and form secondary tumours called metastases. These metastases are the same type of cancer as the primary solid tumour.

The primary solid tumour may be either benign or malignant. For the treatment disclosed herein, the primary solid tumour is typically malignant as these tumours are more likely to metastasize than benign tumours.

The primary solid tumour is to be distinguished from liquid tumours that circulate around the body through the bloodstream.

### Lung Metastases

In the present context, the term “lung metastases” refers to secondary tumours located in the lungs which have spread from a primary solid tumour. Typically, lung metastases are located in the lung parenchyma or pleura.

The terms “lung metastases” and “pulmonary metastases” may be used interchangeably.

### Sarcoma

In the present context, the term “sarcoma” refers to a malignant tumour of transformed cells of mesenchymal origin (connective tissue). The affected mesenchymal cells may be present in different types of connective tissue, such as bone, cartilage, fat, or vascular tissue. Sarcomas may be divided into the two overall groups of bone sarcomas and soft-tissue sarcomas.

Sarcomas are primary solid tumours that may give rise to metastases elsewhere in the body, such as the lungs.

### Melanoma

In the present context, the term “melanoma” refers to a cancer which develops in melanin-producing cells called melanocytes. Melanocytes are a type of mesenchymal cells which are derived from neural crest cells. Accordingly, melanoma is to be considered a sarcoma.

### Carcinoma

In the present context, the term “carcinoma” refers to a malignant tumour of transformed cells of epithelial origin. Carcinomas forms in a tissue that lines the inner or outer surfaces of the body and include, but is not limited to, breast cancer, prostate cancer, lung cancer and colorectal cancer.

Carcinomas may be categorised according to their histological types as adenocarcinoma, squamous cell carcinoma, adenosquamous carcinoma, anaplastic carcinoma, large cell carcinoma, and small cell carcinoma.

In the context of the present treatment, carcinomas are considered primary solid tumours that may give rise to metastases elsewhere in the body, such as the lungs.

### Lymphoma

In the present context, the term “lymphoma” refers to a primary solid tumour that develops from lymphocytes.

### Bacillus Calmette-Guérin (BCG) Composition

In the present context, the term “BCG composition” refers to any composition comprising live attenuated Mycobacterium bovis BCG as active ingredient. The BCG composition may comprise one or more pharmaceutically acceptable carriers, excipients and/or diluents so that it is suitable for administration to a subject, such as a non-human patient. Administration of the BCG composition can be done in nebulized form.

The BCG composition may be a commercially available BCG vaccine.

### Pharmaceutically Acceptable

In the present context, the term “pharmaceutically acceptable” refers to molecular entities and compositions that are suitable for use with animals without undue adverse side effects (such as toxicity, irritation, and allergic response) commensurate with a reasonable benefit/risk ratio.

### Nebulization and Nebulized

In the present context, the term “nebulization” refers to the process of converting a liquid composition to a fine spray, aerosols or mist. Thus, a nebulized composition is in the form of a fine spray, aerosols or mist.

Nebulization may be achieved using a nebulizer, such as a jet nebulizer, ultrasonic nebulizer, or mesh nebulizer, connected to a system for mechanical ventilation.

### Mechanical Ventilation

In the present context, the term “mechanical ventilation” refers to the use of a ventilator to fully or partially provide artificial ventilation.

The terms “mechanical ventilation”, “assisted ventilation” and “intermittent mandatory ventilation” may be used interchangeably.

### Companion Animal

In the present context, the term “companion animal” refers to non-human animals that are domesticated or domestic-bred. Companion animals does not include working animals, livestock or laboratory animals.

Companion animals include, but are not limited to, dogs, cats, horses, rabbits, hamsters, ferrets, and guinea pigs.

The terms “companion animal” and “pet” may be used interchangeably.

### About

Wherever the term “about” is employed herein in the context of amounts, for example absolute amounts, such as numbers, purities, concentrations, weights, sizes, etc., or relative amounts (e.g. percentages, equivalents or ratios), timeframes, and parameters such as temperatures, pressure, etc., it will be appreciated that such variables are approximate and as such may vary by ±10%, for example #5% and preferably ±2% (e.g. #1%) from the actual numbers specified. This is the case even if such numbers are presented as percentages in the first place (for example ‘about 10%’ may mean±10% about the number 10, which is anything between 9% and 11%).

### Treatment of Lung Metastases

The prognosis for companion animals that are unfortunate to develop a cancer are often poor due to limited treatment options or the high cost associated with treatment. The lack of treatment options is even more pronounced for companion animals with metastatic disease, such as lung metastases, for which no effective treatment is available. Thus, companion animals with lung metastases are often euthanized shortly after diagnosis or eventually die from the disease.

Mycobacterium bovis Bacillus Calmette-Guérin (BCG) is a live-attenuated vaccine, initially established to protect against childhood meningitis and disseminated tuberculosis (TB). However, BCG has been demonstrated to hold the potential for increasing the capacity of the immune system to combat other pathogens than TB, e.g. by boosting non-specific responses in both T-cell mediated adaptive responses and innate immune responses.

The inventors of the present invention have devised a method for efficiently directing a BCG composition at lung metastases to unleash the full therapeutic effect of the Mycobacterium bovis.

In particular, it was surprisingly found that administration of the BCG composition directly to the lungs of the companion animal patient caused a substantial reduction of tumour burden. Moreover, it was found that development of further lung metastases was slowed or prevented.

Thus, an aspect of the present invention relates to a Bacillus Calmette-Guérin (BCG) composition for use in the treatment, inhibition or amelioration of lung metastases in a companion animal, wherein the BCG composition is administered to the lungs of a companion animal.

It is to be understood that the administration of BCG to the lungs may also be used as a preventive treatment to prevent development of pulmonary metastases. As such, the BCG composition may be administered upon or after diagnosis of a primary tumour but before development and/or diagnosis of lung metastases.

Therefore, an embodiment of the present invention relates to a Bacillus Calmette-Guérin (BCG) composition for use in the prevention of lung metastases in a companion animal, wherein the BCG composition is administered to the lungs of a companion animal.

Another embodiment of the present invention relates to a Bacillus Calmette-Guérin (BCG) composition for use in the treatment, inhibition, amelioration or prevention of lung metastases in a companion animal, wherein the BCG composition is administered to the lungs of a companion animal.

A further embodiment of the present invention relates to the BCG composition for use as described herein, wherein the treatment of lung metastases results in the reduction or stabilization of the metastases.

Active ingredients intended to assert their effect at a target site in the lungs of patients are typically delivered in the form of aerosols or nebulized compositions. The nebulized composition may be prepared by any conventional means.

Therefore, an embodiment of the present invention relates to the BCG composition for use as described herein, wherein the BCG composition is a nebulized BCG composition.

Another embodiment of the present invention relates to the BCG composition for use as described herein, wherein the BCG composition is nebulized by pneumatic, mechanical or electrical means.

However, delivery to the lungs is complicated in non-human animals who cannot be instructed to breathe through the mouth only. As a consequence, the therapeutic effect of traditional delivery to the lungs by inhalation may be insufficient because much of the active ingredient is retained in the mucosa of the nasal cavity. Moreover, such delivery to non-human animals tends to carry the hazard that the clinician or the animal owner is exposed to the active ingredient which is unwanted.

By delivery of the BCG composition directly to the lungs via intubation, the mucosa of the upper airways (nose, throat, etc.) are bypassed. Without being bound by theory, it is therefore contemplated that a more efficient distribution of the active ingredient is achieved without dispersion of the BCG composition to non-diseased tissues and into the surroundings. It was surprisingly found that this strategy of administration resulted in a clinically detectable response in a canine patient with lung metastases (see Example 1).

Thus, an embodiment of the present invention relates to the BCG composition for use as described herein, wherein the BCG composition is administered to the lungs via a tracheal tube.

Another embodiment of the present invention relates to the BCG composition for use as described herein, wherein the tracheal tube is an endotracheal tube.

A further embodiment of the present invention relates to the BCG composition for use as described herein, wherein the route of administration is orotracheal or nasotracheal, preferably orotracheal.

Mechanical ventilation refers to the use of a ventilator to fully or partially provide artificial ventilation. Non-invasive mechanical ventilation may be accomplished for patients that are conscious and can use a face or nasal mask. Invasive mechanical ventilation relates to the situation wherein placement of an instrument to create an airway that is placed inside the trachea, such as an endotracheal tube or nasotracheal tube, is utilised.

The two main types of mechanical ventilations are positive pressure ventilation in which air is pushed into the lungs through the airways, and negative pressure ventilation in which air is pulled into the lungs. It is contemplated herein that positive pressure ventilation is particularly advantageous for delivery of the BCG composition to pulmonary metastases.

Positive pressure ventilation may be enacted by increasing the patient's airway pressure through an endotracheal tube. The positive pressure allows air to flow into the airway until the ventilator stream is stopped. Then, the airway pressure drops to zero, and the elastic recoil of the chest wall and lungs push the breath out through passive exhalation.

Without being bound by theory, it is contemplated herein that positive pressure ventilation is favourable because the distal areas of the lungs are better inflated and therefore the BCG composition is more completely distributed in the lungs.

Thus, an embodiment of the present invention relates to the BCG composition for use as described herein, wherein the BCG composition is administered under conditions of mechanical ventilation of the companion animal.

A preferred embodiment of the present invention relates to the BCG composition for use as described herein, wherein the BCG composition is administered under conditions of positive pressure ventilation.

Without being bound by theory, it is contemplated that the therapeutic effect of the BCG composition against lung metastases may be caused by its ability to act as an immune stimulating component that efficiently drive maturation of immune cells, such as dendritic cells and macrophages, and mobilise them against the cancerous tissue. The BCG composition may comprise any Mycobacterium bovis strain that induces this maturation, i.e. the present BCG composition is not limited to one particular bacterial strain.

Therefore, an embodiment of the present invention relates to the BCG composition for use as described herein, wherein the BCG composition comprises live attenuated Mycobacterium bovis BCG.

Another embodiment of the present invention relates to the BCG composition for use according to any one of the proceeding items, wherein BCG composition comprises a bacterial strain or sub-strain selected from the group consisting of Pasteur 1173 P2, Danish 1331, Glaxo 1077, Tokyo 172-1, Russian BCG-I, and Moreau RDJ strains.

A further embodiment of the present invention relates to The BCG composition for use as described herein, wherein the attenuated live Mycobacterium bovis BCG is Danish strain 1331.

The BCG composition may function with conventional pharmaceutically acceptable carriers, excipients and diluents and is not limited to any particular one. However, some conventional carriers, excipients and diluents are known to specifically work well with BCG, including, but not limited to, those comprised in BCG vaccines. Thus, the BCG composition may comprise or consist of a commercially available BCG vaccine.

Such vaccines are known to be safe and without serious adverse effects.

Thus, an embodiment of the present invention relates to the BCG composition for use as described herein, wherein the BCG composition comprises one or more pharmaceutically acceptable carriers, excipients and/or diluents.

Another embodiment of the present invention relates to the BCG composition for use as described herein, wherein the one or more pharmaceutically acceptable carriers, excipients and/or diluents are selected from the group consisting of sodium glutamate, magnesium sulphate heptahydrate, dipotassium phosphate, citric acid monohydrate, L-asparagine monohydrate, ferric ammonium citrate, and glycerol.

Other carriers solutions include, but are not limited to, buffered saline solutions, such as Hank's balanced salt solution, Tris buffered saline, HEPES buffered solutions, and others.

Yet another embodiment of the present invention relates to the BCG composition for use as described herein, wherein the BCG composition is a BCG vaccine.

Cancer diseases are notoriously known to be difficult to prognosticate, and the extent of treatment regimens varies from patient to patient. Pulmonary metastases are no different, the required amount of treatment is difficult to predict. However, the vast majority of patients require a repeated administration of effective doses to obtain a positive clinical outcome. The BCG composition herein may be administered repeatedly, if necessary, without any severe adverse effects.

Accordingly, an embodiment of the present invention relates to the BCG composition for use as described herein, wherein the BCG composition is administered as a single effective dose or as multiple effective doses.

Another embodiment of the present invention relates to the BCG composition for use as described herein, wherein the number of effective doses administered is at least 2, such as at least 3, such as at least 4, such as at least 5, such as at least 6, such as at least 7.

A further embodiment of the present invention relates to the BCG composition for use as described herein, wherein each effective dose is ranging from about 0.5×10⁷ to about 2×10⁸ colony forming units (CFU), such as about 1×10⁷ to about 1.5×10⁸ CFU, such as about 2×10⁷ to about 1×10⁸ CFU.

A still further embodiment of the present invention relates to the BCG composition for use as described herein, wherein the concentration of the BCG composition is ranging from about 0.2×10⁷ CFU/mL to about 1×10⁸ CFU/mL, such as from about 0.5×10⁷ CFU/mL to about 8×10⁷ CFU/mL, preferably from about 1×10⁷ CFU/mL to about 4×10⁷ CFU/mL.

The administration of the BCG composition is relatively quick and can be performed dependably when the patient is adequately passivated. Thus, inconsistent administration and dosing caused by anxious or restless companion animals are avoided.

Thus, an embodiment of the present invention relates to the BCG composition for use as described herein, wherein the administration of an effective dose to the lungs is completed within a time period of about 2 min to about 15 min, such as about 5 min to about 10 min.

Another embodiment of the present invention relates to the BCG composition for use as described herein, wherein the companion animal is sedated during the administration of the BCG composition.

Yet another embodiment of the present invention relates to the BCG composition for use as described herein, wherein the companion animal is anaesthetized during the administration of the BCG composition.

Lung metastases are secondary tumours that have spread to the lungs of a subject from another primary tumour site in the body. Primary tumours are most commonly solid and may occur in most tissues of the body, including, but not limited to, bone, breast, lung, prostate, colon, bladder, and kidney. In particular malign primary solid tumours are conducive of metastasizing.

Thus, an embodiment of the present invention relates to the BCG composition for use as described herein, wherein the lung metastases are derived from a primary solid tumour.

All primary tumours may give rise to metastases, independent of the origin of the primary tumour. Thus, lung metastases may be derived from a broad variety of types of cancer.

Accordingly, an embodiment of the present invention relates to the BCG composition for use as described herein, wherein the primary solid tumour is selected from the group consisting of sarcoma, carcinoma, lymphoma, and carcinosarcoma.

Another embodiment of the present invention relates to the BCG composition for use as described herein, wherein the primary solid tumour is a sarcoma selected from the group consisting of melanoma, soft tissue sarcoma, fibrosarcoma, hemangiopericytoma, peripheral nerve sheet tumour, liposarcoma, myxosarcoma, leiomyosarcoma, rhabdomyosarcoma, synovial cell sarcoma, gastrointestinal stromal tumour, histiocytic sarcoma, hemangiosarcoma, lymphangiosarcoma, chondrosarcoma, osteosarcoma, and osteochondrosarcoma.

A preferred embodiment of the present invention relates to the BCG composition for use as described herein, wherein the primary solid tumour is a melanoma.

A further embodiment of the present invention relates to the BCG composition for use as described herein, wherein primary solid tumour is a carcinoma selected from the group consisting of squamous cell carcinoma, nasal adenocarcinoma, thyroid carcinomas and adenocarcinomas, parathyroid carcinomas and adenocarcinomas, gastrointestinal carcinoma and adenocarcinomas, oesophageal carcinoma, gastric carcinoma, small intestinal carcinoma, large intestinal carcinoma, rectal carcinoma, bronchioalveolar carcinomas, pulmonary adenocarcinomas, hepatocellular carcinomas, pancreatic carcinomas and adenocarcinomas, renal cell carcinomas, transitional cell carcinomas, nephroblastoma, prostatic adenocarcinomas, uterine carcinomas and adenocarcinomas, Leydig cell tumour, Sertoli cell tumour, seminoma, granulosa cell tumour, teratocarcinoma, anal gland carcinomas and adenocarcinomas, oligodendrocytoma, glioblastoma, and meningiomas.

At present, companion animals have no viable options for treatment of lung metastases and such a diagnosis is therefore in many cases followed by euthanasia or premature death. It is contemplated that all companion animals will benefit from the treatment scheme described herein. This especially the case since all companion animals lack the degree of consciousness that will allow them to breath only through the mouth and the active ingredient is therefore inclined to get retained in the nasal mucosa upon classical administration via a face mask. Accordingly, the direct administration to the lungs will ensure a better delivery of the BCG composition to the site of lung metastases, even more so when the administration is under conditions of positive pressure ventilation.

Companion animals such as dogs, cats and horses may have a relatively long lifespan for companion animals, and are therefore more likely to contract lung metastases and to benefit from the treatment described herein.

Therefore, an embodiment of the present invention relates to the BCG composition for use as described herein, wherein the companion animal is selected from the group consisting of dog, cat, horse, rabbit, hamster, ferret, and guinea pig.

A preferred embodiment of the present invention relates to the BCG composition for use as described herein, wherein the companion animal is a dog.

Another embodiment of the present invention relates to the BCG composition for use as described herein, wherein the companion animal is a cat.

A further embodiment of the present invention relates to the BCG composition for use as described herein, wherein the companion animal is a horse.

The treatment described herein has been shown to reduce tumour burden of lung metastases. The tumour burden may be evaluated based on several parameters, such as the size of the lung metastases and/or the level of known prognostic immune indicators or biomarkers.

Examples of known tumour burden biomarkers include, but are not limited to, interleukin 8 (IL-8) and interleukin 10 (IL-10). IL-8 is a known biomarker of tumour burden, with high levels of IL-8 being associated with high tumour burden. IL-8 is produced by multiple cancer types, hereunder melanoma, and is involved in protumoural vascularization and inflammation. IL-10 is an anti-inflammatory cytokine. High levels of IL-10 are indicative of tumour-induced immunosuppression.

Therefore, an embodiment of the present invention relates to the BCG composition for use as described herein, wherein administration of the BCG composition causes reduction of tumour burden.

Another embodiment of the present invention relates to the BCG composition for use according to item X29, wherein the reduction of tumour burden is one or more selected from the group consisting of size of lung metastases are reduced, metastases growth rate is slowed, and reduced levels of interleukin-8 (IL-8) and/or interleukin 10 (IL-10) concentration in serum of the companion animal.

As for many other cancer treatments, the present administration of the BCG composition may be combined with administration of another immunotherapeutic composition. The immunotherapeutic composition may be an already commercially available composition or product.

Thus, an embodiment of the present invention relates to the BCG composition for use as described herein, further comprising administration of an immunotherapeutic composition.

Another aspect of the present invention relates to a method of treatment, inhibition or amelioration of lung metastases in a companion animal, the method comprising administration of a BCG composition to the lungs of a companion animal.

The BCG composition may conveniently be provided as kit ready for loading into the intubation system utilized for delivering the BCG composition to the lungs of the companion animal. The intubation system comprises all the components necessary for delivery of the BCG composition to the lungs, including, but not limited to a nebulization chamber for loading the BCG composition into, relevant tubing for intubation of the patient, and a circuit for keeping the patient anaesthetized during administration of the BCG composition.

Accordingly, an aspect of the present invention relates to a kit comprising:
a Bacillus Calmette-Guérin (BCG) composition, and
an intubation system adapted for use with a companion animal.

Another aspect of the present invention relates to an intubation system adapted for use with a companion animal, wherein the intubation system is loaded with a Bacillus Calmette-Guérin (BCG) composition.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the BCG composition, tracheal tube, and mechanical ventilation described in relation to the medical use or method of treatment utilising the BCG composition, which embodiments may readily be part of the kit and intubation system described herein. Embodiments and features of the present invention are also outlined in the following items.

### Items

X1. Bacillus Calmette-Guérin (BCG) composition for use in the treatment, inhibition, amelioration or prevention of lung metastases in a companion animal, wherein the BCG composition is administered to the lungs of a companion animal.
X1a. Bacillus Calmette-Guérin (BCG) composition for use in the treatment, inhibition or amelioration of lung metastases in a companion animal, wherein the BCG composition is administered to the lungs of a companion animal.
X2. The BCG composition for use according to any one of items X1 or X1a, wherein the BCG composition is administered to the lungs via a tracheal tube.
X3. The BCG composition for use according to any one of items X1, X1a or X2, wherein the tracheal tube is an endotracheal tube.
X4. The BCG composition for use according to any one of the preceding items, wherein the route of administration is orotracheal or nasotracheal, preferably orotracheal.
X5. The BCG composition for use according to any one of the preceding items, wherein the BCG composition is administered under conditions of mechanical ventilation of the companion animal.
X6. The BCG composition for use according to any one of the preceding items, wherein the BCG composition is administered under conditions of positive pressure ventilation.
X7. The BCG composition for use according to any one of the preceding items, wherein the BCG composition is a nebulized BCG composition.
X8. The BCG composition for use according to item X7, wherein the BCG composition is nebulized by pneumatic, mechanical or electrical means.
X9. The BCG composition for use according to any one of the proceeding items, wherein the BCG composition comprises live attenuated Mycobacterium bovis BCG.
X10. The BCG composition for use according to any one of the proceeding items, wherein BCG composition comprises a bacterial strain or sub-strain selected from the group consisting of Pasteur 1173 P2, Danish 1331, Glaxo 1077, Tokyo 172-1, Russian BCG-I, and Moreau RDJ strains.
X11. The BCG composition for use according to any one of items X9 or X10, wherein the attenuated live Mycobacterium bovis BCG is Danish strain 1331.
X12. The BCG composition for use according to any one of the proceeding items, wherein the BCG composition comprises one or more pharmaceutically acceptable carriers, excipients and/or diluents.
X13. The BCG composition for use according to item X12, wherein the one or more pharmaceutically acceptable carriers, excipients and/or diluents are selected from the group consisting of sodium glutamate, magnesium sulphate heptahydrate, dipotassium phosphate, citric acid monohydrate, L-asparagine monohydrate, ferric ammonium citrate, and glycerol.
X14. The BCG composition for use according to any one of the proceeding items, wherein the BCG composition is a BCG vaccine.
X15. The BCG composition for use according to any one of the proceeding items, wherein the BCG composition is administered as a single effective dose or as multiple effective doses.
X16. The BCG composition for use according to item X15, wherein the number of effective doses administered is at least 2, such as at least 3, such as at least 4, such as at least 5, such as at least 6, such as at least 7.
X17. The BCG composition for use according to any one of items X15 or X16, wherein each effective dose is ranging from about 0.5×10⁷ to about 2×10⁸ colony forming units (CFU), such as about 1×10⁷ to about 1.5×10⁸ CFU, such as about 2×10⁷ to about 1×10⁸ CFU.
X18. The BCG composition for use according to any one of items X15-X17, wherein the administration of an effective dose to the lungs is completed within a time period of about 2 min to about 15 min, such as about 5 min to about 10 min.
X19. The BCG composition for use according to any one of the proceeding items, wherein the companion animal is sedated during the administration of the BCG composition.
X20. The BCG composition for use according to any one of the proceeding items, wherein the companion animal is anaesthetized during the administration of the BCG composition.
X21. The BCG composition for use according to any one of the proceeding items, wherein the lung metastases are derived from a primary solid tumour.
X22. The BCG composition for use according to item X21, wherein the primary solid tumour is selected from the group consisting of sarcoma, carcinoma, lymphoma, and carcinosarcoma.
X23. The BCG composition for use according to any one of items X21 or X22, wherein the primary solid tumour is a sarcoma selected from the group consisting of melanoma, soft tissue sarcoma, fibrosarcoma, hemangiopericytoma, peripheral nerve sheet tumour, liposarcoma, myxosarcoma, leiomyosarcoma, rhabdomyosarcoma, synovial cell sarcoma, gastrointestinal stromal tumour, histiocytic sarcoma, hemangiosarcoma, lymphangiosarcoma, chondrosarcoma, osteosarcoma, and osteochondrosarcoma.
X24. The BCG composition for use according to any one of items X21-X23, wherein the primary solid tumour is a melanoma.
X25. The BCG composition for use according to any one of items X21 or X22, wherein primary solid tumour is a carcinoma selected from the group consisting of squamous cell carcinoma, nasal adenocarcinoma, thyroid carcinomas and adenocarcinomas, parathyroid carcinomas and adenocarcinomas, gastrointestinal carcinoma and adenocarcinomas, oesophageal carcinoma, gastric carcinoma, small intestinal carcinoma, large intestinal carcinoma, rectal carcinoma, bronchioalveolar carcinomas, pulmonary adenocarcinomas, hepatocellular carcinomas, pancreatic carcinomas and adenocarcinomas, renal cell carcinomas, transitional cell carcinomas, nephroblastoma, prostatic adenocarcinomas, uterine carcinomas and adenocarcinomas, Leydig cell tumour, Sertoli cell tumour, seminoma, granulosa cell tumour, teratocarcinoma, anal gland carcinomas and adenocarcinomas, oligodendrocytoma, glioblastoma, and meningiomas.
X26. The BCG composition for use according to any one of the proceeding items, wherein the companion animal is selected from the group consisting of dog, cat, horse, rabbit, hamster, ferret, and guinea pig.
X27. The BCG composition for use according to any one of the proceeding items, wherein the companion animal is a dog.
X28. The BCG composition for use according to any one of items X1-X26, wherein the companion animal is a cat.
X29. The BCG composition for use according to any one of the proceeding items, wherein administration of the BCG composition causes reduction of tumour burden.
X30. The BCG composition for use according to item X29, wherein the reduction of tumour burden is one or more selected from the group consisting of size of lung metastases are reduced, metastases growth rate is slowed, and reduced levels of interleukin-8 (IL-8) and/or interleukin 10 (IL-10) concentration in serum of the companion animal.
X31. The BCG composition for use according to any one of the proceeding items, further comprising administration of an immunotherapeutic composition.
Y1. A method of treatment, inhibition, amelioration or prevention of lung metastases in a companion animal, the method comprising administration of a BCG composition to the lungs of a companion animal.
Y1a. A method of treatment, inhibition or amelioration of lung metastases in a companion animal, the method comprising administration of a BCG composition to the lungs of a companion animal.
Y2. The method according to any one of items Y1 or Y1a, wherein the BCG composition is administered to the lungs via a tracheal tube.
Y3. The method according to any one of items Y1, Y1a or Y2, wherein the tracheal tube is an endotracheal tube.
Y4. The method according to any one of items Y1-Y3, wherein the route of administration is orotracheal or nasotracheal, preferably orotracheal.
Y5. The method according to any one of items Y1-Y4, wherein the BCG composition is administered under conditions of mechanical ventilation of the companion animal.
Y6. The method according to any one of items Y1-Y5, wherein the BCG composition is administered under conditions of positive pressure ventilation.
Y7. The method according to any one of items Y1-Y6, wherein the BCG composition is a nebulized BCG composition.
Y8. The method according to item Y7, wherein the BCG composition is nebulized by pneumatic, mechanical or electrical means.
Y9. The method according to any one of items Y1-Y8, wherein the BCG composition comprises live attenuated Mycobacterium bovis BCG.
Y10. The method according to any one of items Y1-Y9, wherein BCG composition comprises a bacterial strain or sub-strain selected from the group consisting of Pasteur 1173 P2, Danish 1331, Glaxo 1077, Tokyo 172-1, Russian BCG-I, and Moreau RDJ strains.
Y11. The method according to any one of items Y9 or Y10, wherein the attenuated live Mycobacterium bovis BCG is Danish strain 1331.
Y12. The method according to any one of items Y1-Y11, wherein the BCG composition comprises one or more pharmaceutical acceptable carriers, excipients and/or diluents.
Y13. The method according to item Y12, wherein the one or more pharmaceutically acceptable carriers, excipients and/or diluents are selected from the group consisting of sodium glutamate, magnesium sulphate heptahydrate, dipotassium phosphate, citric acid monohydrate, L-asparagine monohydrate, ferric ammonium citrate, and glycerol.
Y14. The method according to any one of items Y1-Y13, wherein the BCG composition is a BCG vaccine
Y15. The method according to any one of items Y1-Y14, wherein the BCG composition is administered as a single effective dose or as multiple effective doses.
Y16. The method according to item Y15, wherein the number of effective doses administered is at least 2, such as at least 3, such as at least 4, such as at least 5, such as at least 6, such as at least 7.
Y17. The method according to any one of items Y15 or Y16, wherein each effective dose is ranging from about 0.5×10⁷ to about 2×10⁸ colony forming units (CFU), such as about 1×10⁷ to about 1.5×10⁸ CFU, such as about 2×10⁷ to about 1×10⁸ CFU.
Y18. The method according to any one of items Y15-Y17, wherein the administration of an effective dose to the lungs is completed within a time period of about 2 min to about 15 min, such as about 5 min to about 10 min.
Y19. The method according to any one of items Y1-Y18, wherein the companion animal is sedated during the administration of the BCG composition.
Y20. The method according to any one of items Y1-Y19, wherein the companion animal is anaesthetized during the administration of the BCG composition.
Y21. The method according to any one of items Y1-Y20, wherein the lung metastases are derived from a primary solid tumour.
Y22. The method according to item Y21, wherein the primary solid tumour is selected from the group consisting of sarcoma, carcinoma, lymphoma, and carcinosarcoma.
Y23. The method according to any one of items Y21 or Y22, wherein the primary solid tumour is a sarcoma selected from the group consisting of melanoma, soft tissue sarcoma, fibrosarcoma, hemangiopericytoma, peripheral nerve sheet tumour, liposarcoma, myxosarcoma, leiomyosarcoma, rhabdomyosarcoma, synovial cell sarcoma, gastrointestinal stromal tumour, histiocytic sarcoma, hemangiosarcoma, lymphangiosarcoma, chondrosarcoma, osteosarcoma, and osteochondrosarcoma.
Y24. The method according to any one of items Y21-Y23, wherein the primary solid tumour is a melanoma.
Y25. The method according to any one of items Y21 or Y22, wherein primary solid tumour is a carcinoma selected from the group consisting of squamous cell carcinoma, nasal adenocarcinoma, thyroid carcinomas and adenocarcinomas, parathyroid carcinomas and adenocarcinomas, gastrointestinal carcinoma and adenocarcinomas, oesophageal carcinoma, gastric carcinoma, small intestinal carcinoma, large intestinal carcinoma, rectal carcinoma, bronchioalveolar carcinomas, pulmonary adenocarcinomas, hepatocellular carcinomas, pancreatic carcinomas and adenocarcinomas, renal cell carcinomas, transitional cell carcinomas, nephroblastoma, prostatic adenocarcinomas, uterine carcinomas and adenocarcinomas, Leydig cell tumour, Sertoli cell tumour, seminoma, granulosa cell tumour, teratocarcinoma, anal gland carcinomas and adenocarcinomas, oligodendrocytoma, glioblastoma, and meningiomas.
Y26. The method according to any one of items Y1-Y25, wherein the companion animal is selected from the group consisting of dog, cat, horse, rabbit, hamster, ferret, and guinea pig.
Y27. The method according to any one of items Y1-Y26, wherein the companion animal is a dog.
Y28. The method according to any one of items Y1-Y26, wherein the companion animal is a cat.
Y29. The method according to any one of items Y1-Y28, wherein administration of the BCG composition causes reduction of tumour burden.
Y30. The method according to item Y29, wherein the reduction of tumour burden is one or more selected from the group consisting of size of lung metastases are reduced, metastases growth rate is slowed, and reduced levels of interleukin 8 (IL-8) and/or interleukin 10 (IL-10) concentration in serum of the companion animal.
Y31. The method according to any one of items Y1-Y30, further comprising administration of an immunotherapeutic composition.
Z1. A kit comprising:
   a Bacillus Calmette-Guérin (BCG) composition, and
   an intubation system adapted for use with a companion animal.
Z2. The kit according to item Z1, wherein the intubation system comprises a nebulization chamber and a tracheal tube.
Z3. The kit according to any one of items Z1 or Z2, wherein the intubation system comprises a nebulization chamber, a tracheal tube, and an anaesthesia circuit.
W1. An intubation system adapted for use with a companion animal, wherein the intubation system is loaded with a Bacillus Calmette-Guérin (BCG) composition.
W2. The intubation system according to item W1, wherein the intubation system comprises a nebulization chamber and a tracheal tube.
W3. The intubation system according to any one of items W1 or W2, wherein the intubation system comprises a nebulization chamber, a tracheal tube, and an anaesthesia circuit.
W4. The intubation system according to any one of items W1-W3, wherein the nebulization chamber is loaded with the BCG composition.

### EXAMPLES

### Example 1: Treatment of Lung Metastases of a Canine Patient

This example describes treatment of a canine patient (“canine patient X”) with lung metastases and evaluation of the clinical outcome. In particular, nebulized BCG was administered via intubation of the patient.

### Method

### Patient

Canine patient X was an approx. 8 kg mixed breed dog with lung metastases from a solid primary melanoma tumour. The tumour originated in the oral cavity (oral malignant melanoma). The primary tumour was removed surgically (partial mandibulectomy), and the draining lymph nodes were excised. After surgery, canine patient X developed pulmonary metastases.

### Nebulization of BCG:

A BCG solution (Danish strain 1331) was diluted in phosphate buffered saline (PBS) to a desired concentration, calculated according to the weight of the canine patient and approx. 4 mL was used for the procedure (see Table 1 below). The dose of BCG nebulized was escalated from 2.5×10⁷ CFU for the first 2 administrations to 3.5×10⁷ CFU for the 3^{rd} and 4^{th} administrations. For dogs weighing more than 20 kg, doses of double concentration are appropriate.

The reconstituted BCG (BCG nebulization solution) was then aspirated into one single 5 ml syringe and transferred into the nebulization chamber of the nebulizer system (Aerogen® Solo, Hamilton Medical). The nebulizer system comprises the nebulizer, T-piece, optional breathing circuit adapters, and connection cable.

TABLE 1
BCG dose calculations.

| Size of | | | Volume of | Number of | Dose (in 4 |
|---|---|---|---|---|---|
| the dog | #dose | Concentration | PBS/vial | BCG vials | mL of PBS) |
| <20 kg | 1 | 0.625 × 10⁷ | 0.8 mL | 5 | 2.5 × 10⁷ CFU |
| | | CFU/mL | | | |
| | 2 | 0.625 × 10⁷ | 0.8 mL | 5 | 2.5 × 10⁷ CFU |
| | | CFU/mL | | | |
| | 3 | 0.875 × 10⁷ | 0.6 mL | 7 | 3.5 × 10⁷ CFU |
| | | CFU/mL | | | |
| | 4 | 0.875 × 10⁷ | 0.6 mL | 7 | 3.5 × 10⁷ CFU |
| | | CFU/mL | | | |

### Administration of BCG to the Canine Patient

A non-rebreathing T-piece system was used as anaesthesia circuit to deliver the aerosolized BCG. The fresh gas flow was calculated based on canine patient X's size (approximately 200 ml/kg/min).

The canine patient was sedated with butorphanol (0.2 mg/kg intramuscularly) and given 100% oxygen through a mask before anaesthesia was induced by administering propofol intravenously (1-4 mg/kg) over 2 minutes. Once the autonomous reflexes had disappeared, the larynx was visualized using a laryngoscope and an endotracheal tube placed in the trachea. The endotracheal tube cuff was manually inflated with a syringe to ensure no air leakage. The canine patient was then connected to the ventilator, which was then turned on to initiate positive pressure ventilation. Total intravenous anaesthesia (TIVA) was administered (propofol) to maintain a light to medium level of anaesthesia. The patient was ventilated using positive pressure ventilation throughout the procedure.

The canine patient was monitored throughout the procedure using capnography, pulse oximetry, electrocardiogramaod pressure, and temperature. The ventilator was set to ventilate the canine patient with a tidal volume of approximately 10 ml/kg, set to a speed of 10-15 breaths/minute, with a peak inspiratory pressure of 15 cm H₂O (+/−5 cm H₂O), and a peak expiratory pressure of 0 cm H₂O (+/−5 cm H₂O).

Before beginning the administration of BCG, the canine patient received a single dose of diphenhydramine (1 mg/kg intravenously) and acetaminophen (10 mg/kg intravenously).

After transferring the BCG nebulization solution to the nebulization chamber, the nebulization unit was turned on. The operator verified that the BCG solution was being nebulized by visually inspecting the mist filling the ventilation tube. The BCG administration was continued until the nebulization unit ran empty (approx. 4-8 min).

Once the nebulization chamber was empty, the nebulizer was turned off and the canine patient was weaned of the ventilator until it starts breathing spontaneously for 2-3 minutes to clear the circuit of remaining aerosols. The canine patient was then disconnected from the anaesthesia circuit and propofol was discontinued.

As the canine patient began to regain its autonomous reflexes, the endotracheal tube was removed, and the canine patient was monitored until it regained normal functions, and then placed back in the kennel. The canine patient was monitored continuously during this time.

### X-Ray Imaging

Canine patient X was imaged while awake. The thoracic cavity was imaged in four different views: right lateral recumbency, left lateral recumbency, sternal recumbency, and dorsal recumbency. The imaging settings were set according to the patient's weight. Images were assessed by a board-certified veterinary radiologist. The size of pulmonary metastases was assessed subjectively, as well as compared between timepoints using an image analyzing software (Vue PACS v12, Philips).

### CT Scanning

Canine patient X underwent computer tomography (CT) scans on a CT scanner (Revolution™ CT scanner, GE HealthCare) while anesthetized prior to BCG-nebulization. The patient was placed in sternal recumbency. Images were acquired in both soft tissue and bone sequences, before and after administering intravenous contrast solution (Iohexol). The imaging settings were set according to the patient's weight. Images were assessed by a board-certified veterinary radiologist. The size of pulmonary metastases was assessed between timepoints using an image analyzing software (Vue PACS v12, Philips).

### Cytokine Analysis

Serum levels of IL-8 and IL-10 of canine patient X were determined using multiplex analysis (ProcartaPlex™ Canine Cytokine Chemokine Growth Factor Panel 1 11-Plex, Invitrogen). The multiplex analysis was used for:
1. Short term blood sampling. (Between 2 and 96 hrs post BCG nebulization treatment). Analysis of the effect of the BCG nebulization treatment at administration with the aim of detecting a correlation with possible side effects at administration.
2. Short term blood sampling. (Between 2 and 96 hrs post BCG nebulization treatment). Identification of any possible correlation with BCG local administration to the lung and systemic changes in the profile of circulating cytokines.
3. Long term blood sampling. (Blood sampling at baseline, every 2 weeks until the end of the treatment, and then at follow up visits). Identification of any possible correlation between changes in cytokine levels and reduction in tumour burden, e.g. reduction in number and size of metastases.

Briefly, blood samples were acquired from canine patient X, and 0.5 mL blood collected in EDTA-treated tubes was spun down at 1,000×g for 10 min at room temperature. The top layer corresponding to the plasma fraction was then transferred into new tubes and immediately stored at −80° C. until multiplex analysis.

For the multiplex assay, frozen plasma samples were thawed, mixed by vortexing and centrifuged at 10,000×g for 10 min to pellet out particulates. The clear supernatant was then transferred to a new tube and 25 μL of each sample along with standards and blank were plated in duplicate onto a 96 well plate pre-loaded with capture beads, according to manufacturer's instructions. The plate was incubated for 2 hours at room temperature to allow the analytes present in the standards and in the samples to bind to the respective beads. The plate was then washed, 25 μL of the Detection Antibody Mix was added to each well and the plate was incubated with shaking for 30 min at room temperature. The plate was then washed, 50 μL of Streptavidin-PE was added to each well and the plate was incubated with shaking for 30 min at room temperature.

The plate was then washed, 120 μL of Reading Buffer was added and the plate was shaken for 5 min at room temperature. The plate was then inserted into the MAGPIX Luminex instrument (Luminex, DiaSorin) and data were acquired and analysed with the xPONENT software (Luminex, DiaSorin). A graphical presentation of the results was then generated with the GraphPad Prism software.

### Results

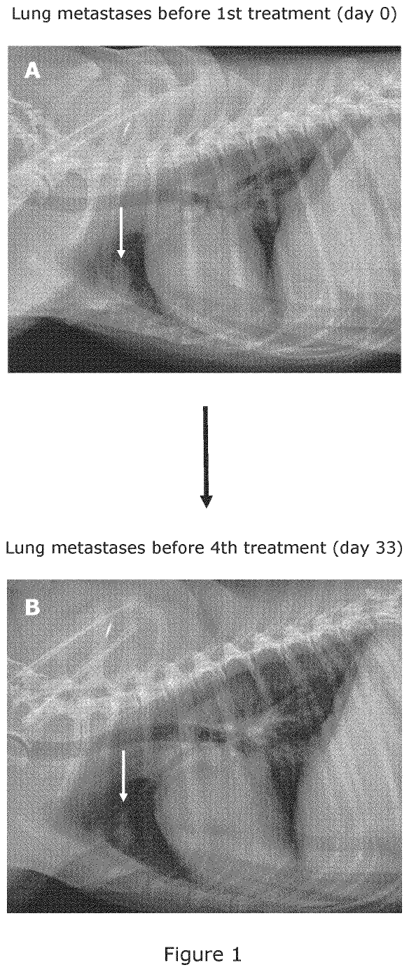

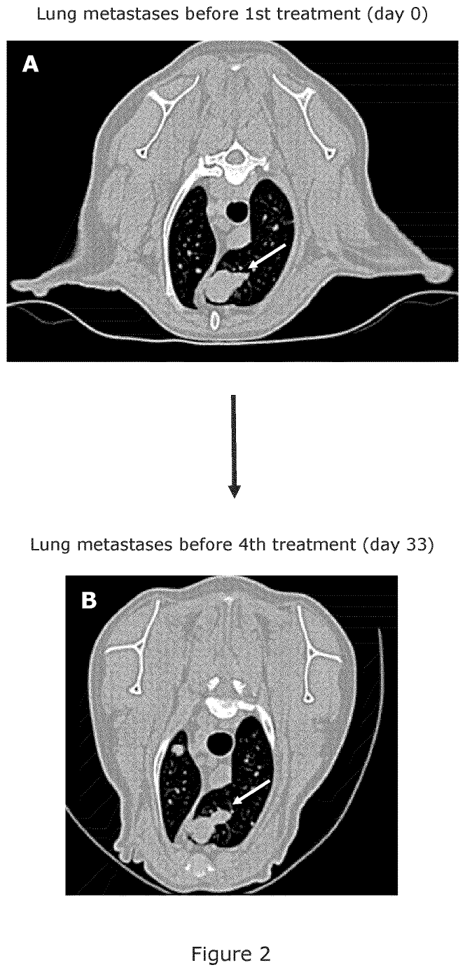

Canine patient X was enrolled for treatment for lung metastases originating from melanoma. X-rays and CT scans were acquired before the treatment started to provide a baseline of disease burden (FIGS. 1A and 2A). Apparent from the x-ray and CT scan at day 0 is a metastasis in the lung of canine patient X (white arrow).

Canine patient X was administered with BCG on days 0, 9, 22, and 33 each administration at a dose concentration of 2.5×10⁷ CFU, 2.5×10⁷ CFU, 3.5×10⁷ CFU, and 3.5×10⁷ CFU, respectively. Administration was performed as described above.

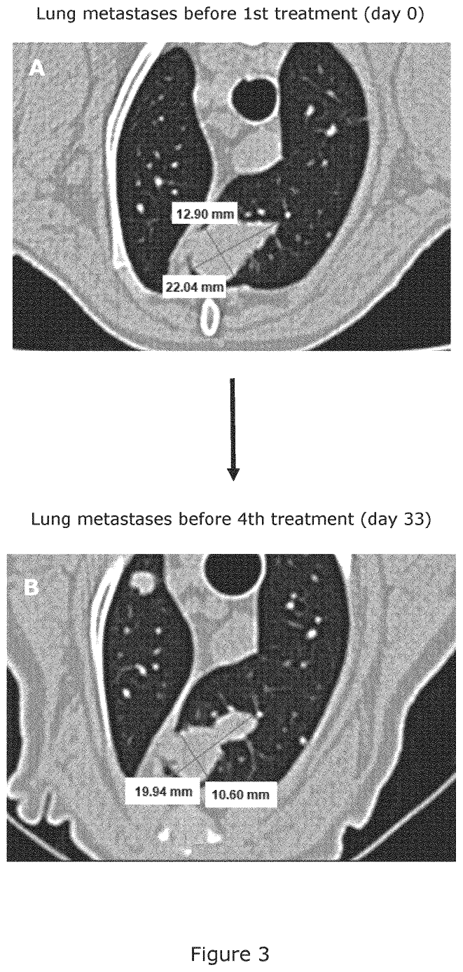

At day 33 (before the 4^{th} administration) x-rays and CT scans were acquired to determine status of the lung metastasis following treatment (FIGS. 1B and 2B). X-ray images show a visual reduction in size of the largest metastasis in the cranial thorax between day 0 and day 33. Similarly, CT-scans show a measurable reduction of the size of the metastasis in the right cranial lung lobe (FIG. 3).

The encouraging reduction in metastasis size was corroborated by measurement of interleukin 8 (IL-8) and interleukin 10 (IL-10) in serum of canine patient X.

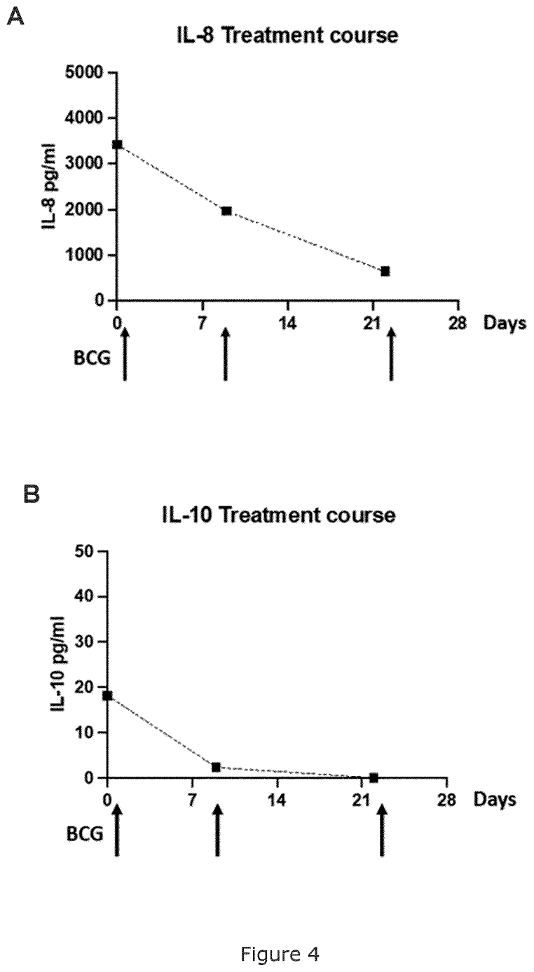

Measurements showed a substantial decrease in both IL-8 (FIG. 4A) and IL-10 (FIG. 4B) levels in serum of canine patient X after commencement of BCG treatment. Thus, the cytokine profile points towards a less immunosuppressed canine patient X with reduced tumour burden following BCG treatment. Moreover, the collated cytokine profiles did not indicate any adverse effects related to the direct administration of BCG to the lungs of canine patient X. Thus, the cytokine data substantiate the positive effect of the treatment.

Canine patient X was euthanized 183 days after being diagnosed with metastases. This significantly surpasses the expected median survival time (MST) for stage IV melanoma of 54 days. Canine patient X was doing very well during treatment and the severity of adverse effects recorded during treatment was less compared to dogs treated with normal chemotherapy. Adverse effects included a mild increase in body temperature (but not defined as fever) the first day after treatment, and mildly increased respiratory rate the day after for some treatments. During the last month before euthanization, canine patient X showed some reduced appetite and activity level. Ultimately, canine patient X was euthanized due to seizures caused by a brain metastasis.

### Conclusion

The treatment of canine patient X provided a clear clinically measurable response to the treatment as demonstrated by a reduction in size of the metastasis and in tumour burden. The canine patient greatly surpasses the expected median survival time.

### Example 2: Multiplex Analysis of Cytokine Response in Canines Treated with BCG Administered by Different Protocols

The aim of the example is to assess the level of inflammatory markers (such as IL-12) in the serum as a function of delivery method of BCG.

### Method

### Administration of BCG

Healthy dogs are treated with an indirect and a direct method of delivery of nebulized BCG. The first treatment scheme involves treatment using the direct method first (intubation and positive pressure ventilation under anaesthesia), followed by an indirect method (spontaneous ventilation while conscious) after 72 hours. The second treatment scheme involves treatment using the indirect method first, followed by the direct method after 72 hours. The same dose of BCG is administered for each of the delivery methods.

The direct method of delivery of nebulized BCG is the same as described in the Example 1. The indirect method of delivery of nebulized BCG is performed in the conscious dog, using an AeroDog-chamber placed over the dog's muzzle and connected to the Aerogen Solo® T-piece. The nebulizer will be run until the chamber is emptied.

### Cytokine Analysis

Serum levels of inflammatory cytokines of the canine patients are determined using the multiplex analysis (ProcartaPlex™ Canine Cytokine Chemokine Growth Factor Panel 1 11-Plex, Invitrogen) as described in Example 1. The blood is sampled at baseline (before nebulization) and between 2 and 24 hours post BCG nebulization, stored at −80° C. and processed for multiplex analyses as described in Example 1.

The levels of inflammatory cytokines, such as IL-12, are determined and compared between treatment schemes.

### Results

It is expected that the level of some inflammatory cytokines, such as IL-12, reach a higher level at 12 hours after the BCG nebulization using the direct method of delivery compared to the indirect methods.

### Conclusion

The direct method of delivery of nebulized BCG induces a more pronounced inflammatory response, suggesting a stronger anti-tumour response.

### Example 3: Treatment of Lung Metastases of a Second Canine Patient

This example describes treatment of a canine patient (“canine patient Y”) with lung metastases and evaluation of the clinical outcome. In particular, nebulized BCG was administered via intubation of the patient.

### Method

### Patient

Canine patient Y was a female Golden retriever of age 3.5 years who was limb amputated due to a chondroblastic osteosarcoma in the distal radius. The osteosarcoma diagnosis was made one month prior to amputation. After limb amputation, canine patient Y received several rounds of chemotherapy with carboplatin.

The prognosis for osteosarcoma diagnosis is very poor without treatment, and even with amputation, most canine patients will have developed massive lung metastases within a few months after amputation. Usually, such patients will be euthanized within six months after diagnosis if no chemotherapy is given, and when given, survival time usually extend to 8-12 months before metastasis have occurred and the dog show signs of metastasis. However, once they have developed metastases, the prognoses is poor, with expected median survival times (MST) varying from 72-89 days depending on treatment.

### Treatment

Canine patient Y received the same treatment as canine patient X, cf. Example 1. Canine patient Y received a total of three BCG nebulizations.

### Results

Canine patient Y was enrolled for treatment for lung metastases originating from the osteosarcoma and received the first BCG nebulization treatment 5 months after the osteosarcoma diagnosis was made. At the time of the first treatment, two distinct metastases in the lungs were visible on CT scans.

At day 71 following the first treatment, and after canine patient Y had received three treatments, CT scans were acquired to determine status of the lung metastasis. The CT scans revealed that no new metastases had appeared.

Unfortunately, canine patient Y did not tolerate anaesthesia well and it was agreed with the owner of the patient that treatment should be discontinued.

At day 101 from the first treatment and diagnosis of the two pulmonary metastases, canine patient Y was euthanized due to poor ambulation and reduced activity level. A new thoracic CT was performed postmortem, showing no new metastases. The clinical symptoms of canine patient Y were not thought to be related to the two pulmonary 5 metastases.

### CONCLUSION

The data on canine patient Y indicate that the BCG nebulization treatment protects against the development of new metastases. Canine patient Y survived substantially longer than the prognosis for the indication (i.e. MST is 72-89 days) and the decision to euthanize the patient was not due to clinical symptoms caused by pulmonary metastases.

1. A method of treating, inhibiting, or ameliorating lung metastasis in a companion animal comprising: administering a composition comprising Bacillus Calmette-Guerin (BCG) to the lungs of a companion animal, which has lung metastasis.

2-20. (canceled)

21. The method according to claim 1, wherein the composition is administered to the lungs via a tracheal tube.

22. The method according to claim 1, wherein the route of administration is orotracheal or nasotracheal.

23. The method according to claim 1, wherein the composition is administered under conditions of mechanical ventilation of the companion animal.

24. The method according to claim 1, wherein the composition is administered under conditions of positive pressure ventilation.

25. The method according to claim 1, wherein the composition is a nebulized composition.

26. The method according to claim 1, wherein the composition comprises a live attenuated Mycobacterium bovis BCG.

27. The method according to claim 1, wherein the composition is administered as a single effective dose or as multiple effective doses.

28. The method according to claim 27, wherein the administration of an effective dose to the lungs is completed within a time period of about 2 min to about 15 min.

29. The method according to claim 1, wherein the lung metastases are derived from a primary solid tumour.

30. The method according to claim 29, wherein the primary solid tumour is selected from the group consisting of sarcoma, carcinoma, lymphoma, and carcinosarcoma.

31. The method according to claim 1, wherein the companion animal is selected from the group consisting of dog, cat, horse, rabbit, hamster, ferret, and guinea pig.

32. The method according to claim 1, wherein the companion animal is a dog or a cat.

33. The method according to claim 1, wherein the administration of the composition reduces the tumour burden.

34. The method according to claim 33, wherein the reduction of tumour burden is one or more selected from the group consisting of size of lung metastases are reduced, metastases growth rate is slowed, and reduced levels of interleukin-8 (IL-8) and/or interleukin 10 (IL-10) concentration in serum of the companion animal.

35. A kit comprising:
a Bacillus Calmette-Guérin (BCG) composition, and
an intubation system adapted for use with a companion animal.

36. The kit according claim 35, wherein the intubation system comprises a nebulization chamber and a tracheal tube.

37. An intubation system comprising a composition, which comprises a Bacillus Calmette-Guérin (BCG).

38. The intubation system according to claim 37, wherein the intubation system comprises a nebulization chamber and a tracheal tube.

39. The intubation system according to claim 37, wherein the nebulization chamber is loaded with the composition.
